(19) United States
(12) Patent Application Publication
(10) Pub. No.: US 2002/0026083 A1
(43) Pub. Date: Feb. 28, 2002

(54) Protective gem-diol group for crossed aldol condensations

(76) Inventor: Christian Everett, Springfield, IL (US)

Correspondence Address: CHRISTIAN EVERETT, 2745 SOUTH 6TH ST. #106, SPRINGFIELD, IL, 62703, US

(21) Appl. No.: 09/910,247

(22) Filed: Jul. 20, 2001

Related U.S. Application Data
(60) Provisional application No. 60/228,955, filed on Aug. 28, 2000.

(51) Int. Cl. C07C 45/45

(52) U.S. Cl. 568/390

(57) ABSTRACT

A process for protecting aldehydes and ketones in crossed aldol condensations comprising protecting the target ketone by forming a gem-diol with ethylene glycol or aldehyde with ethylene glycol or a mono-hydroxy alcohol to allow the additive ketone or aldehyde to condense with the target thereby minimizing by-products, waste and separation difficulties then decomposing the gem-diol with dilute mineral acid.

This process having the advantage that a ketone may be added to a protected aldehyde target which was not previously possible.

### BACKGROUND

1. Field of Invention

This invention relates to aldol condensations, specifically to condensations of two different aldols where one final product is desired.

2. Description of Prior Art

In order to produce new compounds two aldol compounds are often reacted in the known manner. If one of these compounds does not have an alpha-carbon with hydrogen on it (benzaldehyde, formaldehyde, tert-pentaldehyde) the reaction produces the one desired product. However, if both reactants have hydrogen on the alpha-carbon specifity is lost and all four possible products result. Attempts have been made to enhance specifity by increasing the amount of one reactant to 30:1 (U.S. Pat. No. 6,028,231) but this still produces byproducts and waste and is impractical where both reactants are approximately equal in cost.

Also, there is currently no method for a ketone to add to an aldehyde (the ketone losing its carbonyl oxygen and the aldehyde losing its alpha hydrogens), when the compounds are mixed the more reactive aldehyde always adds to the ketone (aldehyde losing carbonyl oxygen, ketone losing alpha hydrogens) producing a completely different compound.

### OBJECTS AND ADVANTAGES

Several objects and advantages of the present invention are:

(A) Increased yield of desired product

(B) Less waste

(C) Ease of separation

(D) Stable under basic conditions of condensation

(E) Decomposed under mildly acidic conditions after condensation

(F) Uses low cost ethylene glycol or mono-hydroxy alcohols

(G) Allows formation of ketone-aldehyde compounds previously available only by many low yield steps

### SUMMARY

In accordance with the present invention a protective gem-diol group for crossed-aldol condensation: whereby the carbonyl oxygen of one of the reactants is rendered unreactive to allow the carbonyl oxygen of the second reactant to react with the alpha-carbon hydrogens of the first reactant

### DESCRIPTION

A typical embodiment of the present invention is to form a gem-diol using ethylene glycol for a ketone and ethylene glycol or a mono-hydroxy alcohol for an aldehyde in the known manner using hydrochloric acid and calcium chloride as co-catalysts and heating at 100 degrees Centigrade for several hours. The gem-diol is separated and introduced into the aldol condensation reaction with the second reactant aldehyde or ketone added gradually in the known manner under basic conditions to form the gem-diol of the desired aldol product.

The gem-diol of the desired aldol product is separated and the gem-diol group is decomposed by heating at 100 degrees Centigrade in dilute aqueous hydrochoric acid which may be mixed with a water-miscible organic solvent.

Alternately, the gem-diol group may be preserved for a time to inhibit polymerization of the product.

### CONCLUSION, RAMIFICATION AND SCOPE

Accordingly the reader will see the gem-diol protective group for crossed aldol condensations is more convenient than current methods. Furthermore the process has additional advantages in that:

yields are increased

by-products and waste are reduced

separation from by-products is easier

allows deactivation of aldehyde carbonyl oxygen so a ketone may add to the aldehyde's alpha-carbon hydrogens

Although the description contains specifitys, these should not be construed as limiting the scope of the invention, but merely providing illustrations of some of presently preferred embodiments of the inventions. For example other glycols may be used or other mineral acids and dehydration agents may be used.

Thus, the scope of the invention should be determined by the appended claims and their legal equivalents rather than by the examples given.

I claim:

1. A method for improving the yield of crossed aldol condensations comprising the steps of:
A: Forming a gem-diol of the reactant losing its alpha-carbon hydrogens in the desired reaction.
B: Adding the reactant losing its carbonyl oxygen in the desired reaction gradually under condensation conditions to the gem-diol of the reactant losing its alpha-carbon hydrogens in the desired reaction to form the gem-diol of the reaction product.
C: Decomposing the gem-diol of the reaction product with dilute mineral acid, in the known manner, whereby the ultimate reaction product is obtained

2. A method for condensing a ketone with an aldehyde whereby the aldehyde loses its alpha-carbon hydrogens and the ketone loses its carbonyl oxygen comprising the steps of:
A: Forming a gem-diol of the aldehyde.
B: Adding the ketone gradually under condensation conditions to the gem-diol of the aldehyde to form the gem-diol of the reaction product.
C: Decomposing the gem-diol of the reaction product with dilute mineral acid, in the known manner, whereby the ultimate reaction product is obtained
